(19) United States
(12) Patent Application Publication
(10) Pub. No.: US 2022/0047152 A1
(43) Pub. Date: Feb. 17, 2022

(54) ENDOSCOPE HAVING EXPANDABLE WORKING CHANNEL

(71) Applicant: Dr. Konstantin Bob, Weinheim (DE)

(72) Inventors: Konstantin Bob, Weinheim (DE); Andreas Gründl, Starnberg (DE)

(21) Appl. No.: 17/415,799

(22) PCT Filed: Dec. 20, 2019
(86) PCT No.: PCT/EP2019/086843

§ 371 Date: Jun. 18, 2021

(30) Foreign Application Priority Data
Dec. 21, 2018 (DE) 10 2018 133 368.4

(51) Int. Cl. A61B 1/018; A61B 1/005; A61B 1/04; A61B 1/00

(52) U.S. Cl. CPC A61B 1/018; A61B 1/00135; A61B 1/04; A61B 1/005

(57) ABSTRACT

An endoscope includes an endoscope body and a working channel for guiding medical tools and/or for the through-flow of media. The working channel extends in the longitudinal direction of the endoscope body and forms a working channel outlet in a distal end portion of the endoscope body. An endoscope head is arranged in a distal end region of the endoscope body and has at least one optical unit. The working channel is partially enclosed in the circumferential direction by a part of the endoscope body that is extensionally rigid in the radial direction, over a predefined circumferential portion, preferably one third to two thirds and more preferably more than half of the total circumference. The endoscope body is expandable at least in parts from a first state having a smaller cross-sectional area to a second state having an enlarged cross-sectional area.

### CROSS-REFERENCE TO RELATED APPLICATION(S)

This application is the United States national phase entry of International Application No. PCT/EP2019/086843, filed Dec. 20, 2019, and claims the benefit of German Application No. 10 2018 133 368.4, filed Dec. 21, 2018. The contents of International Application No. PCT/EP2019/086843 and German Application No. 10 2018 133 368.4 are incorporated by reference herein in their entireties.

### FIELD

The present disclosure relates to an endoscope having an, in particular flexible, tube-shaped or pipe-shaped endoscope body, a working channel extending in the longitudinal direction of the endoscope body for guiding medical tools and/or for the flow of media, said working channel forming a working-channel exit in a distal end portion of the endoscope body, and an endoscope head arranged in a distal end region of the endoscope body, said endoscope head having at least one optical system for imaging/an imaging device. The endoscope body has a proximal shaft, an actively bendable portion distally adjacent to the shaft in the axial direction, and the endoscope head distally adjacent to the actively bendable portion.

### BACKGROUND

In medicine, the use of intracorporeal medical instruments, such as endoscopes, catheters and the like, is quickly increasing for screening, diagnosis and therapy. In order to improve the suitability of these devices for specific applications, they have been optimized in order to serve their purpose in the best possible way. For example, there are optimized endoscopes/gastroscopes for the examination of the esophagus, stomach and duodenum, coloscopes for intestinal examination, angioscopes for blood vessel examination, bronchoscopes for bronchial examination, laparoscopes for examination of the abdominal cavity, arthroscopes for the examination of joints and joint spaces, nasopharygoscopes for the examination of the nasal passage and the pharynx, toroscopes for the examination of the thorax, and intubation scopes for the examination of a person's airway.

In medical applications, conventional endoscopes have a tube-shaped or pipe-shaped endoscope body that is connected at its proximal end to a handle or control element. The endoscope body is adapted to be inserted into a patient's body cavity in order to perform a selected therapeutic or diagnostic procedure. The endoscope body also has an imaging device (e.g., with optical fibers extending along the length of the endoscope body, or a CCD/CMOS system) and can provide access for irrigation, suction, tissue grasping, or other functions. In the prior art, the endoscope body is usually sized such that it houses or forms one or more internal working channels that extend along the endoscope body. The working channels are adapted to receive conventional endoscopic equipment, such as minimally invasive instruments for performing surgical procedures. Since the working channel is located within the endoscope body or endoscope shaft, the maximum working channel size is limited by the diameter of the endoscope body or of the endoscope shaft and by the space requirements of the other endoscopic elements running through the endoscope body/endoscope shaft. There are standardized working channel diameters, e.g. 2.8 or 3.8 mm in gastroscopes or 4.2 mm in duodenoscopes.

Endoscopes with a flexible shaft often have an actively bendable or deflectable distal shaft portion, which is also called deflecting portion. The distal deflecting portion of the shaft of a prograde (straight-looking) flexible endoscope with a deflectable tip usually consists of hinged ring elements which form the support structure of the shaft and are operated and tilted against each other by Bowden cables, often called bending control cables. In order to facilitate insertion into the cavity and to prevent the penetration of substances, the ring elements are surrounded by a flexible shell made of a plastic material. In particular, light and image guide cables, channels for fluids or endoscopic working instruments run inside the ring elements. The bending control cables are guided along the outside or inside of the ring elements. Such flexible endoscopes are disclosed, for example, in U.S. Pat. Nos. 6,270,453 B1, 6,482,149 B1 or DE 101 43 966 B4.

The minimally invasive instruments used with such actively bendable endoscopes are usually dimensioned in such a way that they are barely compatible with the common working channel diameters. In particular with tight radii of curvature, the problem arises that the working channels, which already have narrow dimensions, no longer provide sufficient pivoting space for a minimally invasive instrument. While smaller, more compact, minimally invasive instruments are generally desirable, in practice this is often either technically unfeasible or the nature of the therapeutic treatment (e.g., the size of a specimen to be removed) excludes the use of smaller instruments. Often, even larger minimally invasive surgical instruments would be beneficial for treatment, but cannot be transported to the treatment site due to the constructional limitations of the existing endoscopes.

### SUMMARY

In view of the disadvantages of the prior art described above, it is the object of the present invention to provide an endoscope which allows greater freedom in the selection and construction of compatible, minimally invasive surgical instruments and which reliably transports such minimally invasive surgical instruments around tight radii of curvature.

An endoscope according to the invention has at least one tube-shaped or pipe-shaped endoscope body and a working channel running along the longitudinal direction of the endoscope body on or in the latter for guiding medical tools and/or for the flow of media, said working channel forming a working-channel exit in a distal end portion of the endoscope body. The endoscope body has a proximal shaft, an actively bendable portion distally adjacent to the shaft in the axial direction, and an endoscope head distally adjacent to the actively bendable portion. Accordingly, the endoscope head (or an end cap) is disposed in a distal end portion of the endoscope body and includes at least one imaging device. According to the invention, the working channel is at least in sections encompassed in the circumferential direction by a part of the endoscope body, which is stretch-stiff in the radial direction, over a predetermined circumferential portion. In other words, the working channel is limited over the predetermined circumferential portion by the stretch-stiff part of the endoscope body, so that the working channel cannot be radially expanded over the predetermined circumferential portion. Preferably, the predetermined circumferential portion is one third to two thirds and further preferably more than half of the total circumference of the working channel. Preferably, the working channel is at least partially, for example also completely, arranged within the endoscope body. In other words, the working channel is not formed by a working channel on the outside of/externally attached to the endoscope body. According to the invention, the working channel is expandable, at least in sections, from a first (base) state with a smaller cross-sectional area to a second state with an increased cross-sectional area, in order to allow the insertion of differently dimensioned tools. In other words, according to the present invention, the pipe-like working channel of an endoscope is constructed in such a way that at least its inner cross-section (its inner diameter) is kept passively or actively expandable in order to create space, at least temporarily, for the passage of a minimally invasive instrument or tool that exceeds the inner cross-section/inner diameter of the working channel in its (non-expanded) base state. For this purpose, the working channel may be adapted either in its material properties and/or structurally to be expanded. If the working channel lies within the endoscope body (shaft, deflecting portion, head), the endoscope body is also structurally adapted to expand with the working channel or to create space for an expansion of the working channel.

The advantage of the arrangement according to the invention is that instruments which have a diameter that exceeds the inner diameter of the working channel in its base state, either completely or in sections, can be guided through it to their destination by at least temporarily widening the working channel. In addition, instruments configured for the inner diameter of the working channel in its base state can be guided in an improved manner around narrow radii of curvature. Thus, in particular, a temporarily expandable working channel can be integrated into actively bendable endoscopes, which are formed to be comparatively (stretch-) stiff in order to ensure bending, without increasing the overall diameter of the endoscope. Since the working channel remains in its base state when the endoscope is first inserted, the endoscope according to the invention can be configured with the same overall diameter as a comparable conventional endoscope. In addition, encompassing of the part of the endoscope body, which is stretch-stiff in the radial direction, around the predetermined circumferential portion of the working channel offers the advantage that it cannot expand radially over the entire circumference of the working channel. This reduces the risk that the cross-section of the working channel can be narrowed, for example by bending, or even that the working channel can be squeezed off, since the working channel is supported over the predetermined circumferential portion.

According to a preferred configuration example, the working channel can be arranged running along an outer surface of the endoscope body. Preferably, a guide groove running in the longitudinal direction of the endoscope body can be provided in a lateral surface of the endoscope body, said guide groove supporting the working channel radially on the inside and holding or guiding it in a form-fit manner in the circumferential direction of the endoscope. In other words, the working channel can be formed as an area-stretchable tube separate from the endoscope body and guided along the outside of the latter. Due to the external arrangement, expandability can be provided with comparatively little effort, since no major adaptations to the endoscope body per se are necessary.

According to another preferred embodiment, the endoscope body may have at least two segments extending in its longitudinal direction, which are movable relative to each other in the radial and/or circumferential direction. Preferably, one of these segments may be a base segment, in the interior of which various functional lines, Bowden cables and the like run, while the second segment, which is movable relative to the base segment, forms an expandable wall/boundary/guide for the working channel.

According to a preferred further development, the separation plane/interface between the two segments runs in such a way that they can be relatively moved from a first state with a smaller cross-sectional area of the endoscope body to a second state with an increased cross-sectional area of the endoscope body. The two segments form a channel between them running in the longitudinal direction of the endoscope body, which can either form the expandable working channel per se or in which a tube-shaped working channel can be arranged. It could also be said that the working channel can be arranged between the two relatively movable segments.

According to a further aspect, the relatively movable segments may also move relative to each other, e.g., by concentrically rotating in the axial direction of the endoscope, such that the cross-sectional area of the endoscope body does not increase but rather such that the endoscope body opens to one side so that the working channel can expand outwards through said opening. In other words, one of the relatively movable segments may be transferable in the manner of a sliding door into a position overlapped with the at least one other segment, thereby creating a window or recess in the side wall of the endoscope body through which the working channel can expand (bulge out).

According to a preferred configuration example, the endoscope body may include a first base segment and a second pivot segment pivotally connected to the base segment parallel to the longitudinal axis of the base segment, wherein the second pivot segment defines a substantially circular cross-section of the endoscope body together with the base segment in a first position and is transferable to a second position pivoted away from the base segment.

According to a further preferred aspect, the pipe-shaped or tube-shaped endoscope body may comprise, at least in sections, a spring-elastic outer wall that is slotted in its longitudinal direction or which is rolled in its cross-section.

According to a further preferred embodiment, the endoscope body or the working channel may have a helically or spirally wound, spring-elastic outer wall. Preferably, the axial ends of the helically or spirally wound, spring-elastic outer wall can first be twisted against each other in such a way that the inner diameter of the spiral/helix is reduced, and can be installed in this pre-stressed state. Therein, the axial ends are secured against relative rotation and fixed in this state. The anti-rotation mechanism can then be released during use so that the spirally wound, spring-elastic outer wall assumes a relaxed state of larger cross-sectional area or larger diameter as a result of the pre-stressing. Alternatively, a distal portion of the spirally wound, spring-elastic outer wall can be configured to be rotatable relative to a proximal portion, e.g. via a flexible shaft, so that an expansion of the spiral can be activated by a relative rotation against the winding direction of the spiral. Preferably, the helically or spirally wound, spring-elastic outer wall can be coated/sheathed internally and/or externally with a plastic to provide a smooth, fluid-tight outer wall.

According to a preferred embodiment, the working channel may be formed to be tube-shaped and area-stretchable to allow stretching in its longitudinal and transverse directions. Preferably, the stretchability can be elastic in nature so that the working channel returns to its base state after passing a minimally invasive instrument of larger diameter through it.

According to a preferred configuration example, the working channel can have at least one wall layer which is formed as a textile tube and thus provides a structural expandability (by relative movement of individual fibers or threads/yarns). Preferably, the textile tube may be in the form of a woven fabric, meshed fabric, knitted fabric, braided fabric or a net.

According to a preferred further development, the textile tube may contain portions of elastic fibers, such as elastane, which provide area-elastic properties of the textile. Further preferably, fibers with tensile strength/tensile stiffness (e.g., aramid fibers) may be incorporated into the textile with a certain amount of play, forming an automatic, structural stretch limit at a predetermined stretch. Preferably, the textile tube (inside and/or outside) can be coated or covered with a plastic, in particular an elastomer, in order to provide a smooth surface and, if applicable, fluid tightness.

According to a further preferred aspect, the working channel may have an expandable wall structure in the manner of a stent, e.g. with a lattice structure or meandering webs. Such a stent-like working channel may preferably be enveloped in a smooth, closed shell or covered with a film.

According to a further aspect, at least a portion of the endoscope body, in particular a distal portion of the endoscope body, may have an expandable outer wall structure in the manner of a stent. For example, the endoscope head may be plastically expandable in the manner of a stent in order to provide an increased range of motion at a treatment site.

According to a further preferred aspect, the working channel may be made from an area-elastic (solid) material in a tube-shape. Elastomeric materials, in particular silicone and polyurethanes, are preferred.

According to a preferred configuration example, the endoscope body and—if located externally—the working channel may be sheathed in a protective shell which is area-stretchable. The protective shell, like the working channel, may preferably be made of a textile tube or an elastomeric material according to one of the aforementioned aspects. In particular, the protective shell serves to provide a common smooth surface of the endoscope body (of the segments of the endoscope body) and the working channel, which improves insertability of the endoscope. In addition, the protective shell prevents body fluids from entering the endoscope.

### BRIEF DESCRIPTION OF THE DRAWING FIGURES

Hereinafter, configuration examples of the present disclosure are described based on the accompanying figures. In this regard:

FIG. 1 shows a perspective view of an endoscope according to a first embodiment of the invention;

FIG. 2 shows a cross-sectional view through the endoscope according to the first embodiment in a base state;

FIG. 3 shows a cross-sectional view through the endoscope according to the first embodiment with an expanded working channel;

FIG. 4 shows a perspective view of an endoscope according to a second embodiment of the invention;

FIG. 5 shows a cross-sectional view through the endoscope according to the second embodiment in a base state;

FIG. 6 shows a cross-sectional view through the endoscope according to the second embodiment with an expanded working channel;

FIG. 7 shows a cross-sectional view through an endoscope according to a third embodiment in a base state;

FIG. 8 shows a cross-sectional view through the endoscope according to the third embodiment with an expanded working channel;

FIG. 9 shows a front view of a closing mechanism for the endoscope according to the third embodiment;

FIG. 10 shows a perspective view of an endoscope according to a fourth embodiment of the invention;

FIG. 11 shows a perspective view of an endoscope according to a fifth embodiment of the invention;

FIG. 12 shows a cross-sectional view through the endoscope according to the sixth embodiment in a base state;

FIG. 13 shows a cross-sectional view through the endoscope according to the sixth embodiment with an expanded working channel;

FIG. 14 shows an exemplary embodiment for an expandable working channel with a textile layer;

FIG. 15 shows an exemplary embodiment for an expandable working channel with a spiral-shaped spring reinforcement; and

FIG. 16 shows a second exemplary embodiment for an expandable working channel with a spiral-shaped spring reinforcement.

### DETAILED DESCRIPTION

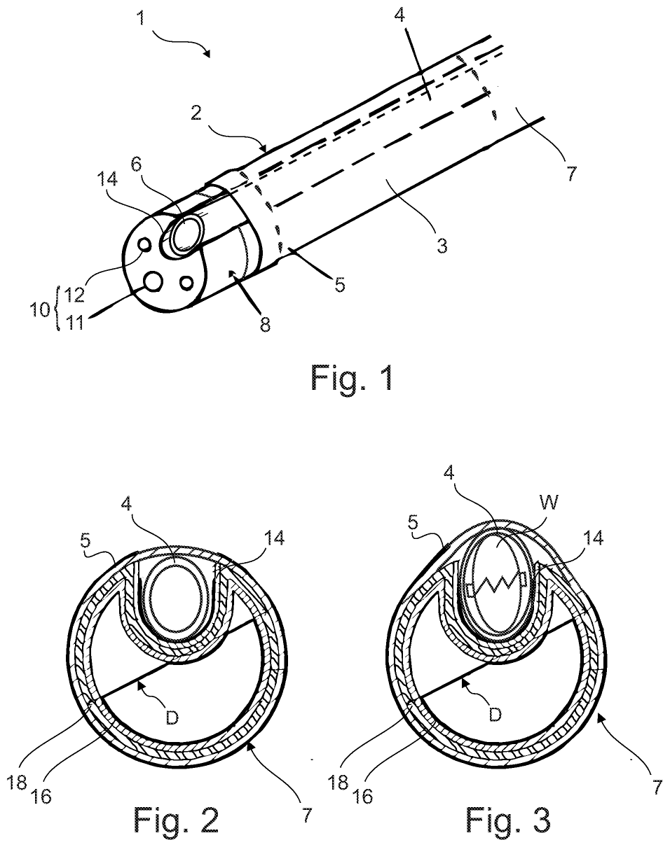

FIG. 1 shows an endoscope according to a first embodiment of the present invention in a perspective view. The endoscope 1 shown has a tube-shaped, shear-stiff, flexibly bendable endoscope body 2, with which it can be inserted into a body cavity. At the proximal (close to the user) end, the endoscope body 2 is provided with a handle part or control element (not shown). The handle part is equipped with a number of operating elements, for example with operating elements for an actively bendable portion 3 (deflecting portion) of the endoscope 1, flushing and suction switches, light switches or the like. The flushing and suction switches enable flushing fluid to be transported to a treatment site within the body or fluids to be suctioned off via a tube-like working channel 4 of the endoscope 1, said working channel 4 extending from the handle part in the longitudinal direction of the endoscope body 2 up to its distal end to form a working-channel exit 6 there. The working channel 4 also allows minimally invasive surgical instruments or tools (W in FIG. 3) to be placed at the treatment site in a targeted manner.

The endoscope 1 shown in FIG. 1 has an endoscope head 8 at its distal (facing away from the user) end portion, with an imaging device 10 including an objective lens 11 and lamps 12 (here LEDs) being arranged on or in said endoscope head 8. The objective lens 11 projects an image onto a CCD/CMOS system (not shown) arranged inside the head. A plurality of lines, not further shown, run inside the endoscope body, with said lines, among other things, transmitting the images captured by the imaging device 10 into the proximal direction to a base station (not shown) or the like.

Usually as well as in the following configuration examples, the endoscope body 2 therefore has at least one proximal (passively) flexible shaft 7, an actively bendable portion 3 distally adjacent to this in the axial direction, and an endoscope head 8 arranged distally at or to the actively bendable portion 3. The working channel 4 runs in or along the endoscope body 2 and usually ends with its working channel exit 6 at the level of the endoscope head 8.

The endoscope 1 shown according to the first configuration example has the aforementioned actively bendable portion 3 proximally adjacent to the endoscope head 8. In the configuration example shown, the actively bendable portion 3 is formed of a number of annular or vertebra-like segments which are not shown in more detail and which are sequentially arranged one after the other in the longitudinal direction of the endoscope body and which can be tilted relative to each other and which can be actuated (tilted relative to each other), e.g. via a Bowden-cable mechanism, from the handle part. The angular deflection achieved by said actively bendable portion 3 can lead to problems in the endoscopes known in the prior art when minimally invasive surgical instruments W are pushed through the working channel 4. Usually, the minimally invasive surgical instruments W are configured in such a way that they can just barely be guided through the common working channel diameters. However, since the minimally invasive surgical instruments W often have longer, stiff devices such as clamps or scissor mechanisms at their distal end, it is geometrically impossible to guide the tools around the curve defined by the working channel 4 above a certain curvature.

The illustrated endoscope 1 according to the first configuration example of the invention provides a remedy to this problem, as the working channel 4 is formed as a tube made of an area-stretchable, here an area-elastic, material which will be explained in more detail later. In endoscopes known from the prior art, the working channel is usually formed as a rigid channel within the endoscope body 2 (within the flexible shaft 7, the actively bendable portion 3 and the endoscope head 8). In order to be able to use the area-stretchability of the expandable working channel 4 according to the invention, it is arranged in the embodiment shown in FIG. 1 outside/on the outside of the endoscope body 2 along its longitudinal direction and is supported on its circumferential lateral surface.

In the preferred embodiment shown, the endoscope body 2 of the endoscope 1 has a guide groove 14 that extends in its longitudinal direction through the lateral surface. In this way, the working channel 4 is guided or fixed to the endoscope body 2 in the circumferential direction. A flexible, area-elastic shell 5 covers the endoscope body 2 together with the working channel 4 and thus ensures on the one hand that the two components have a common, smooth outer contour, which facilitates the insertion of the endoscope 1. In addition, the shell 5 secures the working channel 4 in the guide groove 14 through its inherent elasticity.

FIG. 2 shows a cross-section through the endoscope 1 according to the first preferred configuration example in the region of the flexible shaft 7 of the endoscope 1. In its base state, the endoscope 1, as shown, has a substantially circular outer cross-section enclosed by the shell 5 with the diameter D. The flexible shaft 3 per se (and also the actively bendable portion 3 as well as the endoscope head 8), on the other hand, has a crescent-shaped cross-section due to the guide groove 14, which is designed here with a U-shaped cross-section. FIG. 2 also clearly shows that the flexible shaft 7 has a layered structure. The background to this is that endoscope shafts have to meet a wide range of technical requirements, such as high flexibility, tensile/(transverse) compressive strength, torsional strength, fluid tightness, etc. In detail, the shaft 7 has a first, shear-stiff, flexibly bendable layer 16, e.g. of interlocked metal profiles or plastic profiles, and a second layer 18, encompassing the first layer, for providing the necessary tightness and compressive strength, e.g. a wire-reinforced or fiber-reinforced plastic layer. Other constructions of the flexible shaft 7 common in the prior art are also possible and conceivable. In the base state of the endoscope 1 shown in FIG. 2, the working channel 4 lies in the guide groove 14 and is secured by the shell 5. Likewise, if the shell 5 is appropriately configured, it is also possible to omit the separate tube-shaped working channel 4 so that the working channel 4 is formed by the space between guide groove 14 and shell 5.

FIG. 3 shows the same arrangement as FIG. 2 with the difference that a tool W is pushed through the working channel 4, the diameter of which exceeds the diameter of the working channel 4 in the base state. Due to the aforementioned arrangement, the area-elastic working channel 4 is supported radially outwards only by the likewise area-elastic shell 5, so that these two components 4, 5 can expand radially outwards to create space for the tool W. Towards the central axis of the endoscope 1, the working channel 4 fits snugly against the U-shaped profile of the guide groove 14 and can be supported in this direction on the flexible shaft 7.

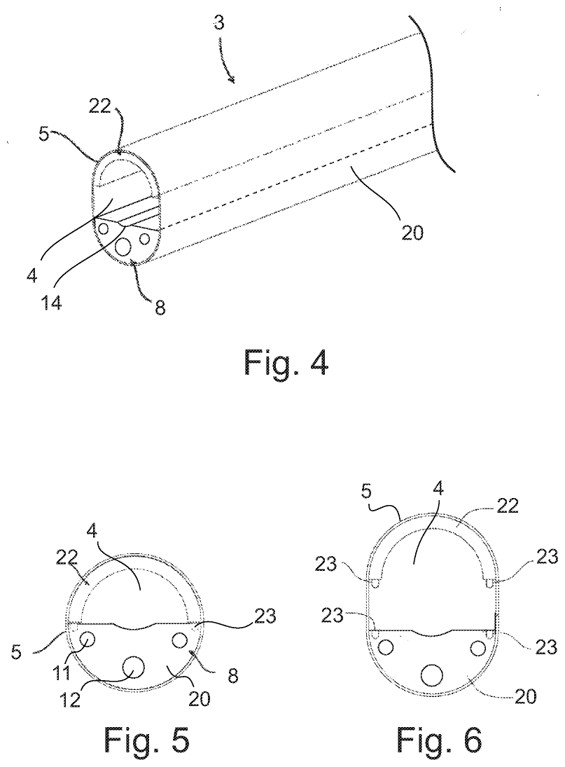

FIG. 4 shows a second preferred embodiment of the invention in an expanded state. The endoscope 1 according to the second embodiment has many similarities with the endoscope of the first embodiment. Thus, this endoscope 1 also has an endoscope head with an imaging device 10, an actively bendable portion 3 and a flexible shaft 7. In contrast to the endoscope of the first embodiment, however, the working channel 4 in the endoscope 1 according to the second embodiment does not run outside the endoscope body 2. Instead, the endoscope body 2 or its cross-section is divided into two relatively movable segments 20, 22 extending in its longitudinal direction.

FIG. 5 shows a front view of the endoscope head 8 of the endoscope 1 according to the second embodiment of the invention in its base state. In this configuration, the endoscope 1 resembles conventional, flexible endoscopes in that it forms an endoscope body 2 with a substantially circular cross-section, inside which the working channel 4 runs. In contrast to conventional endoscopes, however, the endoscope body 2 shown has a separation plane parallel to the longitudinal axis, so that the U-shaped working channel wall segment 22 extending in the longitudinal direction of the endoscope can move away from a base segment 20 of the endoscope body in the radial direction of the endoscope 1, as shown in FIG. 6, in order to expand the working channel 4. On its side/surface facing the working channel wall segment 22, the base segment 22 has a guide groove 14 for guiding the direction of advance of minimally invasive tools. Both segments 20, 22 are encompassed by the area-elastic shell 5.

The segments 20, 22, of the endoscope body 2 have complementary guiding geometries 23 at their interface, which engage with each other in the base state to bring about a form fit between the segments 20, 22 in the transverse direction to the predetermined/desired direction of expansion of the working channel 4.

It is of course possible and contemplated, analogously to the first configuration example, to use a separate, tube-shaped working channel 4 made of an area-stretchable or area-elastic material within the channel formed between the segments 20, 22.

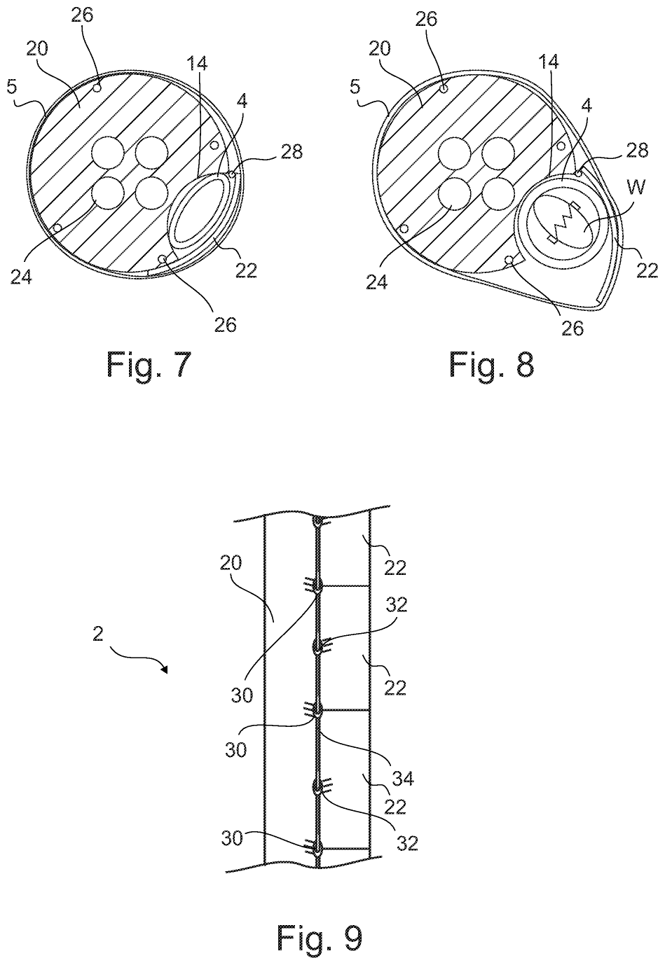

FIG. 7 shows a third configuration example of the present invention. A cross-section through a vertebra-like segment of an actively bendable portion 3 of said endoscope 1 according to the third embodiment is shown. A number of channels 24 for carrying data transmission lines, flushing fluid or the like run within said segment. Also formed in the edge region of the actively bendable portion 3 are axially running Bowden cable channels 26, through which a Bowden cable, not shown here, runs via which the individual vertebra-like segments can be tilted against each other.

The third embodiment of the invention shown in FIGS. 7 and 8 also has an expandable working channel 4. Analogously to the second embodiment of the invention, the working channel extends internally between a first base segment 20 of the endoscope body 2, in which the aforementioned channels 24, 26 run, and, being movable in the radial direction relative thereto, a working channel wall segment 22. In the third embodiment of the invention, the working channel wall segment 22 is pivotally connected to the base segment 20 via a hinge device 28. In a base state in which the working channel wall segment 22 abuts the base segment 20, the two segments 20, 22 together form a substantially circular cross-section of the endoscope body 2. In this base state, the working channel wall segment 22 forms a radial outer cover for a guide groove 14 running in the longitudinal direction in the lateral surface of the base segment 20. In the embodiment shown, the tube-shaped, area-elastic working channel 4 is arranged between the two segments 20, 22 and running within the guide groove 14. However, the segments 20, 22 could just as well also form the cavity lying between them per se as the working channel.

Preferably, the working channel wall segment 22 can be pre-stressed in the base position. For this purpose, the hinge device 28, for example, can be spring-loaded. The shell 5 can also contribute to pre-stressing the two segments 20, 22 in their base position via an internal area elasticity.

FIG. 9 shows a closing mechanism for an endoscope 1 according to the aforementioned third embodiment of the invention. In the embodiment shown, the working channel wall segment 22 is also subdivided in the longitudinal direction of the endoscope into a plurality of segments 22, each of which is pivotally connected to the base segment 20. The closing mechanism has a series of first eyelets 30 associated with the base segment 20 and has a series of second eyelets 32 associated with the working channel wall segments 22. The eyelets 30, 32 are arranged to run in the longitudinal direction of the endoscope. An elongated closing means 34, for example a wire, a string, a thin rod or the like is threaded through the eyelets 30, 32 and holds them in a form fit in their relative position. By pulling out the closing means in the proximal direction, the edges of the working channel wall segments 22 facing away from the hinge device 28 are released so that the space between them and the base segment 20 can expand.

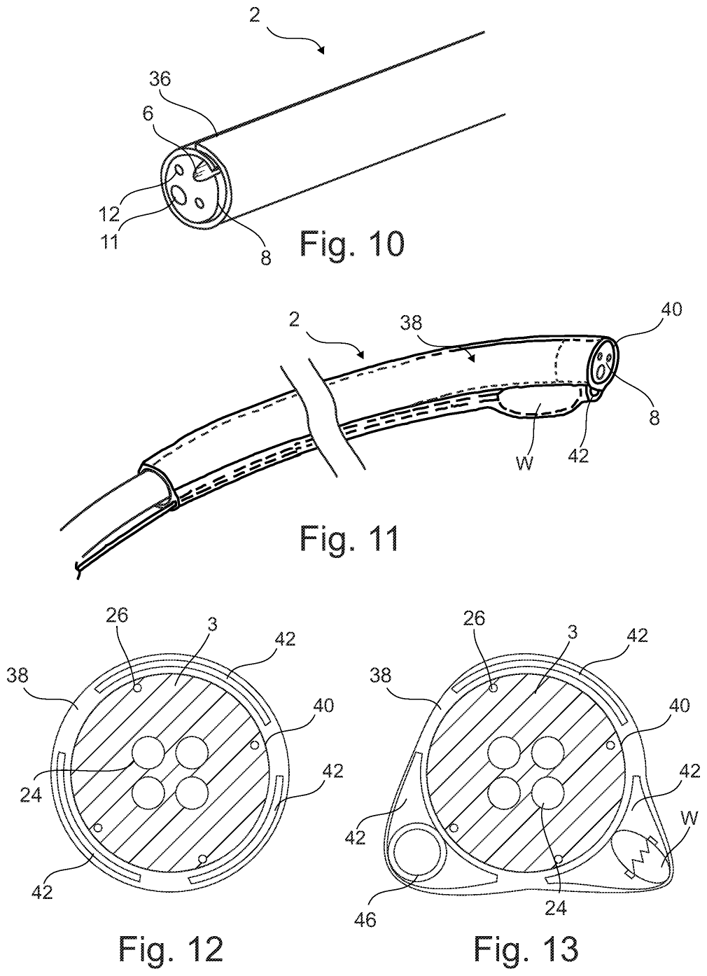

FIG. 10 shows a fourth configuration example of the invention. In this example, an endoscope 1 with a guide groove 14 in its lateral surface is provided as already described in the first configuration example (cf. FIGS. 1 to 3). The shown endoscope according to the fourth embodiment is additionally surrounded by a rigid, rolled, outer circumferential-wall layer 36. In use, the outer wall layer 36 is again covered by a shell 5, which is hidden here for the sake of better illustration. In the embodiment shown, the outer wall layer 36 is made of a thin spring steel sheet, but the use of adapted plastics is also possible. Analogously, an elastic pipe slit in the longitudinal direction can also be used as outer wall layer 36. An outer wall layer 36 according to one of the variants described can be configured to be more rigid than closed pipe or tube structures, since the overlapping (or, in the case of the slotted pipe, opposite) free end edges of the rolled/slotted structure can perform a relative movement in the circumferential direction when a tool with a larger diameter is pushed through.

FIG. 11 shows a fifth embodiment of an endoscope 1 with expandable working channel 4. In the configuration example shown, the endoscope 1 is covered by a multi-lumen tube 38. Specifically, the multi-lumen tube has a main lumen 40 in which the flexible shaft 6, the actively bendable portion 3, and the endoscope head 8 are arranged. The multi-lumen tube 38 further comprises a working-channel lumen 42 that runs parallel to the main lumen 40 and is adjacent to its circumference in order to form the working channel 4. The multi-lumen tube 38 shown is made of a material with area-elastic properties and good sliding properties, e.g. a polyurethane or a silicone. FIG. 11 clearly shows how the working-channel lumen 42 expands to create space for a large-diameter portion of a minimally invasive surgical tool W. According to the preferred configuration example, the multi-lumen tube 38 is formed of a material with nonlinear elastic or nonlinear viscoelastic properties, i.e., at a certain degree of stretching of the material, the stress-stretching curve increases exponentially and the material thus forms an intrinsic stretching limit by its properties.

FIGS. 12 and 13 show in cross-sectional view a sixth embodiment of the invention, which is a further development of the fifth embodiment. As can be clearly seen in the illustrations, the multi-lumen tube can also form several (here three) working-channel lumens 42, which are circumferentially distributed adjacent to a central main lumen 40. In this way, for example, a suction pipe 46 can be placed in one working-channel lumen 42, while a tool W is placed in a second working-channel lumen 42.

The expandable working channel 4 and/or the shell 5 of all embodiments can preferably be formed from nonlinear area-elastic material as already explained.

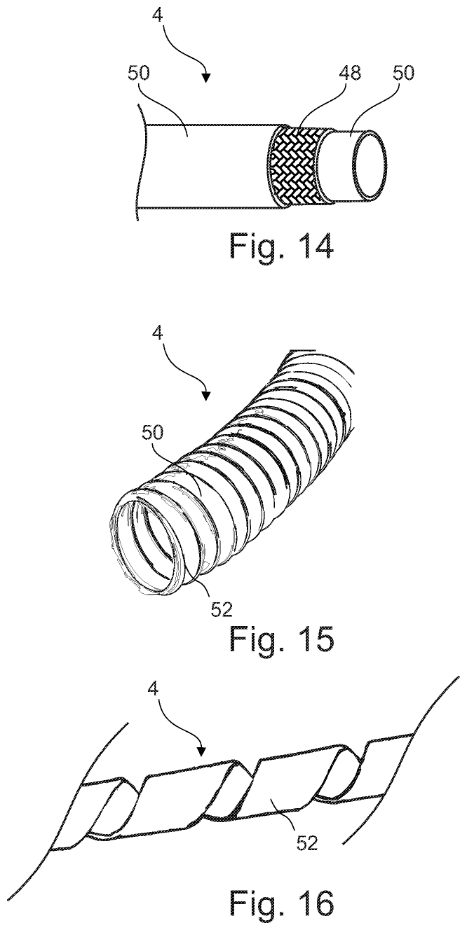

Alternatively, the working channel 4 and/or the shell 5 may also be configured as a textile tube-shaped structure or have at least one textile layer. FIG. 14 shows an example of a working channel 4 that has such a textile tube-shaped layer 48. Particularly suitable are woven fabric, knitted fabric, meshed fabric, braided fabric or nets which already provide structural expansibility/expandability due to the relative mobility of the threads. Particularly preferably, the textile can basically be made of fibers/threads with elastic properties, into which additional tensile-stiff, flexibly bendable threads, e.g. made of aramid, are incorporated with a certain amount of play so that they form a stretch limitation. In the case of a textile configuration of the working channel 4 and/or of the shell, a thin elastomer layer 50 should preferably be applied on the inside and/or outside in order to provide a smooth surface for better sliding properties as well as fluid tightness, as is also shown in FIG. 14. The textile tube layer 48 has the advantage that it is possible to adjust a sufficient elasticity for expandability with sufficient stiffness for guiding minimally invasive instruments as well as an integrated yield limit/puncture resistance.

FIG. 15 shows another preferred configuration example for an expandable working channel 4. The working channel 4 shown is reinforced by a coil spring 52, which forms the working channel 4 in conjunction with an elastomer layer 50.

FIG. 16 shows another preferred embodiment of the working channel 4, which like the previous embodiment is reinforced by a coil spring 52. In this embodiment, however, the coil spring 52 is flatter or in the form of a spirally wound strip. The band-shaped coil spring 52 can be twisted to form a closed tube with a base diameter which is pre-stressed. The two axial ends (not shown) are first fixed to each other around the longitudinal axis of the working channel 4 so that they cannot rotate. In order to expand the working channel, the rotational fixation is removed. Under pre-stressing of the inherent elasticity of the coil spring 52, it assumes the expanded state shown in FIG. 16 with an enlarged diameter compared to the basic diameter. A working channel with such a coil spring 52 is also preferably covered with an elastomer layer, which has been omitted in FIG. 16 for clearer presentation. An outer wall construction for a working channel 4 according to FIG. 16 can also be used for an outer wall of an endoscope body 2.

Based on the configuration examples described, numerous variants are conceivable. For example, the expandability of the working channel 4 and/or of shell 5 does not have to be area-elastic but could just as well be caused by a plastic deformation/stretching of the material. It is important that the smaller working-channel diameter is present in the base state when the endoscope is inserted. Removal of an endoscope with a plastically expanded working channel is usually possible without any problems.

1. An endoscope comprising a tube-shaped or pipe-shaped endoscope body, which has a shaft, an actively bendable portion distally adjacent to the shaft in an axial direction, and an endoscope head distally adjacent to the actively bendable portion, the endoscope head comprising at least one imaging device, and a working channel running in a longitudinal direction of the endoscope body for guiding medical tools and/or for a flow of media, the working channel forming a working-channel exit in a distal end region of the endoscope body,
the working channel being partially encompassed in a circumferential direction by a part of the endoscope body which is stretch-stiff in a radial direction over a predetermined circumferential portion, and which is expandable at least in sections from a first state with a smaller cross-sectional area into a second state with an increased cross-sectional area for for introduction of differently dimensioned tools.

2. The endoscope according to claim 1, wherein the working channel is arranged running along an outer surface of the endoscope body.

3. The endoscope according to claim 1, wherein
the endoscope body comprises at least two segments extending in the longitudinal direction that are movable relative to each other in the radial and/or circumferential direction, so that the at least two segments are movable relative to each other from the first state of smaller cross-sectional area into the second state of increased cross-sectional area; and
the working channel is formed by an intermediate space of the at least two segments or as a tube-shaped or pipe-shaped separate element running between the at least two segments.

4. The endoscope according to claim 3, wherein the at least two segments comprise a base segment and a wall segment of the working channel pivotably hinged to the base segment along a longitudinal axis thereof, said wall segment defining, in a first position, a substantially circular cross-section of the endoscope body together with the base segment and being transferable into a second position pivoted away from the base segment.

5. The endoscope according to claim 1, wherein the endoscope body has a spring-elastically expandable outer wall which is slotted in the longitudinal direction or which is rolled in its cross-section.

6. The endoscope according to claim 1, wherein the working channel is formed to be tube-shaped and area-stretchable to allow stretching in its longitudinal direction and in its transverse direction.

7. The endoscope according to claim 6, wherein the working channel has at least one wall layer which is formed as a textile tube.

8. The endoscope according to claim 7, wherein the textile tube comprises elastic fibers for providing an area-elasticity and comprises additional tensile strength fibers which form a stop or a stretch limitation after a predetermined degree of expansion of the working channel or a predetermined degree of stretching of the textile tube.

9. The endoscope according to claim 6, wherein the working channel is formed to be tube-shaped from an area-elastic material.

10. The endoscope according to claim 1, wherein the endoscope body is sheathed or covered by an area-stretchable protective shell with a smooth outer surface.

11. The endoscope according to claim 1, wherein the part of the endoscope body which is stretch-stiff in the radial direction encompasses one third to two thirds of a total circumference of the working channel in the circumferential direction.

12. The endoscope according to claim 1, wherein the part of the endoscope body which is stretch-stiff in the radial direction encompasses one half of a total circumference of the working channel in the circumferential direction.

13. The endoscope according to claim 2, wherein the working channel is arranged in a guide groove running in the longitudinal direction.
